(19) United States
(12) Patent Application Publication
(10) Pub. No.: US 2019/0054254 A1
(43) Pub. Date: Feb. 21, 2019

(54) LIQUID FLOW DETERMINATION AND CONTROL IN A HYPODERMIC NEEDLE DEVICE

(71) Applicant: QUALCOMM Incorporated, San Diego, CA (US)

(72) Inventors: Paul Robert HOFFMAN, San Diego, CA (US); Eugene DANTSKER, San Diego, CA (US); Robert GANTON, San Diego, CA (US); John Earl AMSCHLER, Del Mar, CA (US); Brian NIZNIK, San Diego, CA (US); Robert BALLAM, Eatons Hill (AU)

(21) Appl. No.: 16/104,031

(22) Filed: Aug. 16, 2018

Related U.S. Application Data
(60) Provisional application No. 62/547,063, filed on Aug. 17, 2017.

(51) Int. Cl. A61M 5/32; A61M 5/24

(52) U.S. Cl. CPC A61M 5/329; A61M 5/2466; A61M 2205/3334; A61M 2205/50; A61M 2005/3128; A61M 2205/3584; A61M 5/3202; A61M 2205/18; A61M 2205/3379

(57) ABSTRACT

Techniques are disclosed for providing a flowmeter in a hypodermic needle device to measure the flow of a drug from a reservoir located within a hypodermic needle device through a needle assembly. In some embodiments a valve may be additionally provided to control the flow of the drug from a reservoir located within a hypodermic needle device through a needle assembly. Such functionality can help ensure the proper dosage is dispensed and/or determine when the drug is improperly dispensed.

### RELATED APPLICATIONS

This application claims the benefit of U.S. Provisional Application No. 62/547,063, filed Aug. 17, 2017, entitled “LIQUID FLOW DETERMINATION AND CONTROL IN A HYPODERMIC NEEDLE DEVICE”, of which is assigned to the assignee hereof, and incorporated herein in its entirety by reference.

### BACKGROUND

### Background Field

The subject matter disclosed herein relates to hypodermic needles, and more particularly to techniques for measuring and controlling liquid flow in a hypodermic needle device.

### Relevant Background

In the field of drug administration, ensuring the drug to be administered is of primary importance. This may be especially true in situations where a drug is self-administered by the patient (rather than by a healthcare professional). Problematically, however, it can sometimes be difficult to ensure accuracy in a dosage using traditional devices that deliver drugs via hypodermic needle. And such devices rarely are able to control the flow of a liquid drug to help ensure an accurate dosage is dispensed from the device and administered to the patient.

### SUMMARY

Embodiments disclosed herein use a flowmeter and (optionally) a valve to measure and (optionally) control the flow of a drug from a reservoir located within a hypodermic needle device through a needle assembly. Such functionality can help ensure the proper dosage is dispensed and/or determine when the drug is improperly administered.

An example hypodermic needle device, according to the description, comprises a body and a flowmeter secured by the body of the hypodermic needle device such that, when a drug is being dispensed by the hypodermic needle device, the flowmeter is located between a reservoir of the drug and a needle assembly. The flowmeter is further configured to take a measurement of an amount of the drug that flows from the reservoir of the drug through the needle assembly. The hypodermic needle device further comprises a processor communicatively coupled with the flowmeter and configured to receive, from the flowmeter, the measurement of the amount of the drug that flows from the reservoir of the drug through the needle assembly.

Alternative embodiments of the hypodermic needle device may comprise one or more of the following features. The flowmeter may comprise a Coriolis flow sensor. The Coriolis flow sensor may be configured to take a measurement of a density of the drug and communicate the measurement of the density of the drug to the processor, and the processor may be configured to, based at least in part on the measurement of the density of the drug received from the Coriolis flow sensor, determine a type of the drug dispensed. The hypodermic needle device may further comprise a flow valve communicatively coupled with the processor and located within the hypodermic needle device such that, when the drug is being dispensed by the hypodermic needle device, the flow valve is located between the reservoir of the drug and the needle assembly. The processor may be configured to operate the flow valve to stop administration of the drug based at least in part on a triggering event. The triggering event may comprise a determination that a predetermined amount of a dosage has been dispensed, a determination that a wrong drug is being dispensed based at least in part on a density measurement taken by the flowmeter, or any combination thereof. The processor may be configured to operate the flow valve using a voltage to control a flow rate of the drug when a drug is being dispensed. The body of the hypodermic needle device may be further configured to house a removable cartridge, wherein the removable cartridge stores the reservoir of the drug. A body of the flowmeter maybe shaped to pierce a seal of the removable cartridge when the removable cartridge is inserted into the body of the hypodermic needle device.

An example method of dispensing a drug with a hypodermic needle device, according to the description, comprises take a measurement, with a flowmeter of a hypodermic needle device located between a reservoir of the drug and a needle assembly, of an amount of the drug that flows from the reservoir of the drug through the needle assembly. The method further comprises communicating data indicative of the measurement to a processor of the hypodermic needle device.

Alternative embodiments of the method may include one or more the following features. The flowmeter may comprise a Coriolis flow sensor. The method may further comprise measuring, with the Coriolis flow sensor, a density of the drug, communicating the measurement of the density of the drug to the processor of the hypodermic needle device, and determine, with the processor of the hypodermic needle device, a type of the drug dispensed, based at least in part on the measurement of the density of the drug. The hypodermic needle device may further comprise a flow valve communicatively coupled with the processor and located within the hypodermic needle device such that, when the drug is being dispensed by the hypodermic needle device, the flow valve is located between the reservoir of the drug and the needle assembly. The method may further comprise operating the flow valve to stop administration of the drug based at least in part on a triggering event. The triggering event may comprise a determination that a predetermined amount of a dosage has been dispensed, or a determination that a wrong drug is being dispensed, based at least in part on a density measurement taken by the flowmeter, or any combination thereof. Operating the flow valve may comprise using a voltage to control a flow rate of the drug when a drug is being dispensed. A body of the hypodermic needle device may be further configured to house a removable cartridge, and the removable cartridge may store the reservoir of the drug. A body of the flowmeter may be shaped to pierce a seal of the removable cartridge when the removable cartridge is inserted into the body of the hypodermic needle device.

An example hypodermic needle device, according to the description, comprises measuring means configured to take a measurement of an amount of a drug that flows from a reservoir of the drug within the hypodermic needle device through a needle assembly of the hypodermic needle device. The measuring means is located between the reservoir the needle assembly. The hypodermic needle device further comprises processing means configured to obtain data indicative of the measurement from the measuring means.

Alternative embodiments of the hypodermic needle device may include one or more the following features. The measuring means may comprise a Coriolis flow sensor. The measuring means may be configured to measure a density of the drug, and the processing means may be configured to obtain the measurement of the density of the drug and determine a type of the drug dispensed, based at least in part on the measurement of the density of the drug. The hypodermic needle device may further comprise flow restriction means communicatively coupled with the processing means and located within the hypodermic needle device such that, when the drug is being dispensed by the hypodermic needle device, the flow restriction means is located between the reservoir of the drug and the needle assembly. The hypodermic needle device may further comprise means for operating the flow restriction means to stop administration of the drug based at least in part on a triggering event. The triggering event may comprise a determination that a predetermined amount of a dosage has been dispensed, a determination that a wrong drug is being dispensed, based at least in part on a density measurement taken by the measuring means, or any combination thereof. The means for operating the flow restriction means may comprise means for using a voltage to control a flow rate of the drug when a drug is being dispensed. The hypodermic needle device may further comprise means for housing a removable cartridge that stores the reservoir of the drug. The hypodermic needle device may further comprise means for piercing a seal of the removable cartridge when the removable cartridge is inserted into the hypodermic needle device.

An example non-transitory computer-readable medium, according to the description, comprises instructions embedded thereon for dispensing a drug with a hypodermic needle device. The instructions comprise computer code for taking a measurement, with a flowmeter of a hypodermic needle device located between a reservoir of the drug and a needle assembly, of an amount of the drug that flows from the reservoir of the drug through the needle assembly, and communicating data indicative of the measurement to a processor of the hypodermic needle device.

Alternative embodiments of the non-transitory computer-readable medium may comprise one or more the following features. The instructions may comprise computer code for operating a flow valve to stop administration of the drug based at least in part on a triggering event. The instructions may further comprise computer code for determining the triggering event, where the instructions for determining the triggering event comprise instructions for determining that a predetermined amount of a dosage has been dispensed, or instructions for determining that a wrong drug is being dispensed, based at least in part on a density measurement taken by the flowmeter, or any combination thereof.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is an example system for providing information about the administration of medicine by a hypodermic needle device to one or more stakeholders.

FIG. 2 is an exploded view of an pen injector, according to an embodiment.

FIG. 3 is a cross-sectional view of a pen injector, according to an embodiment.

FIG. 4 is a block diagram illustrating the of components of a hypodermic needle device 110, according to an embodiment.

FIGS. 5A and 5B are simplified cross-sectional diagrams of a portion of a hypodermic needle device having a flowmeter, according to embodiments.

FIG. 6 is a flow diagram illustrating a method of dispensing a drug with a hypodermic needle device, according to an embodiment.

### DETAILED DESCRIPTION

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

Hypodermic needle devices (e.g., injector pens, auto injectors, syringe needles, etc.) dispense liquid drugs into the body of a patient (e.g., directly into a muscle, vein, or other location under a patient's skin) by pushing a volume of the drug from a chamber or cylinder within the device through a hypodermic needle that (typically) has been injected into the skin of patient. Oftentimes these drugs may be self-administered by the patient, such as when the drugs are administered in an emergency (as may be the case with epinephrine, for example) or frequently administered (as may be the case with insulin, for example).

Establishing that the right dose of the right drug is administered to the right patient at the right time via the right route can be important not only to the person taking the drug, but to many other entities as well. Other stakeholders that have an interest in this information include, for example, the doctor that prescribed and/or is overseeing the administration of the drug, a manufacturer of the drug, an insurance provider (and/or other payer), a government health agency and/or other health organization, and/or the like. For each of the stakeholders, the use and/or misuse of a drug may impact the decisions of a stakeholder with regard to the drug. For example, a drug that is consistently misused may impact whether or how an insurance provider is willing to pay for the use of the drug, and/or how to adjust premiums for patients that may consistently misuse drugs. It may also prompt a manufacturer to modify the means of administration of the drug to help reduce the misuse of its administration. All stakeholders may be impacted by use/misuse information in determining how effective a drug for a population of patients.

Embodiments disclosed herein help increase the accuracy of this information provided to the stakeholders by using a flowmeter to measure an actual volume of the amount of drug dispensed to the patient. This information can be used and/or combined with other information to help determine whether to dispense the drug and/or whether the drug has been administered properly (e.g., whether an accurate dosage was dispensed or not). Additionally, a valve may be utilized to help ensure an accurate dosage is dispensed. Additional details are provided herein below.

It can be noted that, although embodiments described herein are directed toward a hypodermic needle device, embodiments are not so limited. Techniques utilizing a flowmeter (and optionally a valve) in the manner described herein can be utilized in any of a variety of other devices (e.g., syringes, inhalers, etc.) in which liquid or gas flow occurs, which may or may not have medical applications.

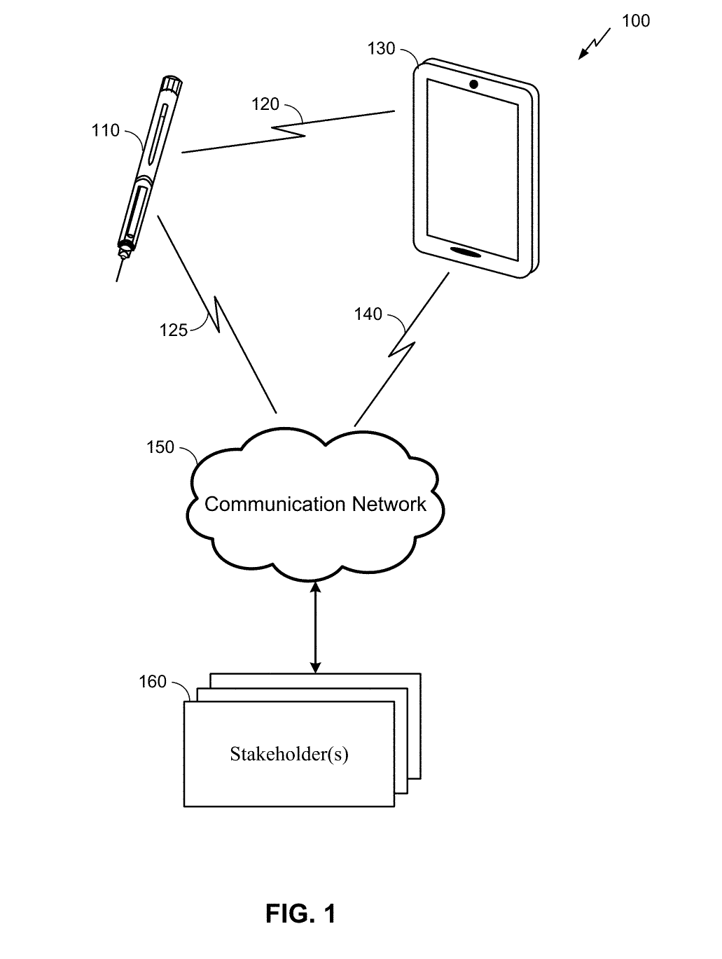

FIG. 1 is an example system 100 for providing information about the administration of medicine by a hypodermic needle device 110 to one or more stakeholders 160. Here, the system 100 may comprise the hypodermic needle device 110 as described herein, along with a connecting device 130, communication network 150, and the stakeholder(s) 160. It will be understood, however, that embodiments of a system 100 may include a different configuration of components, the addition and/or omission of various components, and/or the like, depending on desired functionality. Moreover, it will be understood that techniques described herein may be utilized in a hypodermic needle device 110 that may not necessarily be part of a larger system, such as the system 100 illustrated in FIG. 1.

The hypodermic needle device 110, which is described in more detail herein below, is used to dispense a drug to a patient. Here, a person (e.g., a doctor, nurse, or patient him/herself) may administer the drug by engaging a physical mechanism (e.g., pressing down on a plunger, actuating automatic injection with a button, etc.) while a needle of the hypodermic needle device 110 is injected into the patient's skin. In some embodiments, once the drug is dispensed, the hypodermic needle device 110 can then register, store and transmit data associated with the administration of the drug to the connecting device 130. This data can be transmitted wirelessly via a communication link 120, using any of a variety of wireless technologies as described in further detail below. That said, some embodiments may additionally or alternatively utilize wired communication.

The connecting device 130 may comprise any of a variety of electronic devices capable of receiving information from the hypodermic needle device 110 and communicating information to the stakeholder(s) 160 via the communication network 150. This can include, for example, a mobile phone, tablet, laptop, portable media player, personal computer, or similar device. In some embodiments, the connecting device 130 may comprise a specialized device utilized for purposes of conveying information from the hypodermic needle device 110 (and possibly other medical devices) to the stakeholder(s) 160. In some embodiments, the connecting device 130 may comprise a device owned and operated by the patient (e.g., the patient's mobile phone). In other embodiments, the connecting device 130 may be owned and/or operated by another entity, such as a healthcare provider, insurance company, government agency, etc.

The connecting device 130 may execute an application to provide the data processing and/or relaying functionality illustrated in FIG. 1. In some embodiments, the application may be configurable by a user, or may simply be downloaded to the connecting device 130 and executed automatically. The application may help establish the communication link 120 between the hypodermic needle device 110 and the connecting device 130, which may or may not require input from the user, depending on desired functionality. In some embodiments, the application may provide instructions to a user on proper use of the hypodermic needle device 110 and/or feedback to a user when improper use of the hypodermic needle device 110 is detected. Additional and/or alternative functionality of an application executed by the connecting device 130 may be utilized as desired. (Such functionality may include simple relaying of the data to a remote destination or interacting with the patient about the drug administration such as confirmation and user feedback.)

The communication network 150 may comprise any of a variety of data communication networks, depending on desired functionality. The communication network 150 can include any combination of radio frequency (RF), optical fiber, satellite, and/or other wireless and/or wired communication technologies. In some embodiments, the communication network 150 can comprise the Internet and/or different data networks may comprise various network types, including cellular networks, Wi-Fi® networks, etc. These types may include, for example, a Code Division Multiple Access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, Wideband-CDMA (W-CDMA), and so on. Cdma2000 includes IS-95, IS-2000, and/or IS-856 standards. A TDMA network may implement Global System for Mobile Communications (GSM), Digital Advanced Mobile Phone System (D-AMPS), or some other RAT. An OFDMA network may employ LTE (including LTE category M (Cat-M) or 5G), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. The communication network 150 may additionally or alternatively include a wireless local area network (WLAN), which may also be an IEEE 802.11x network, and a wireless personal area network (WPAN) may be a Bluetooth network, an IEEE 802.15x, Zigbee® network, and/or some other type of network. The techniques described herein may also be used for any combination of wireless wide area network (WWAN), WLAN and/or WPAN.

The communication link 140 between the connecting device 130 and the communication network 150 can vary, depending on the technologies utilized by these components of the system 100. For embodiments where the connecting device 130 is a mobile phone, for example, the communication link 140 may comprise a wireless communication link utilizing the mobile phone's cellular or Wi-Fi® functionality. In embodiments where the connecting device 130 is a personal computer, for example, the communication link 140 may comprise a wired communication link that accesses the communication network 150 via a cable or digital subscriber line (DSL) modem.

It can be noted that some embodiments may not utilize a connecting device 130 to relay data to the communication network 150. In such embodiments, the hypodermic needle device 110 may connect directly to the communication network 150 (as shown in FIG. 1 by communication link 125, which may be used in addition to or as an alternative to communication link 120). For example, the hypodermic needle device 110 may comprise a Long Term Evolution (LTE) category M (Cat-M) device, NarrowBand IoT (NB-IoT), or other Low Power Wide Area Network (LPWAN). Additionally or alternatively, the hypodermic needle device 110 may comprise wireless technology similar to the corresponding functionality of the connecting device 130 described above. In such embodiments, the communication network may additionally or alternatively comprise a Bluetooth Mesh network (such as CSRMesh), a Wi-Fi network, Zigbee, or WWAN (such as LTE, including Cat-M, or 5G). In some embodiments, the hypodermic needle device 110 may connect both with the communication network 150 via communication link 125 and with the connecting device 130 the communication link 120. In such embodiments, the connecting device 130 may not need to separately communicate information regarding the hypodermic needle device 110 to stakeholders 160, but instead the hypodermic needle device 110 may communicate this information directly to the stakeholders 160 via the communication network 150.)

As noted above, the stakeholder(s) 160 may include any of a variety of entities with an interest in the proper administration of medicine by the hypodermic needle device 110. This can include an individual practitioner (e.g., a doctor or nurse), a hospital, a drug manufacturer, an insurance provider (or other payer), a government agency or other health organization, and/or the like. In some embodiments, the user of the hypodermic needle device 110 (e.g., the patient) may also be a stakeholder 160 to which information regarding the use of the hypodermic needle device 110 is provided. Governmental health regulations and/or legal agreements between the patient and/or the stakeholder(s) 160 may apply to the dissemination of information regarding the administration of a drug by the hypodermic needle device 110 to the stakeholder(s) 160. Here, as mentioned above and described in further detail below, the hypodermic needle device 110 can utilize a flowmeter to help ensure the accuracy of the information disseminated to the stakeholder(s) 160.

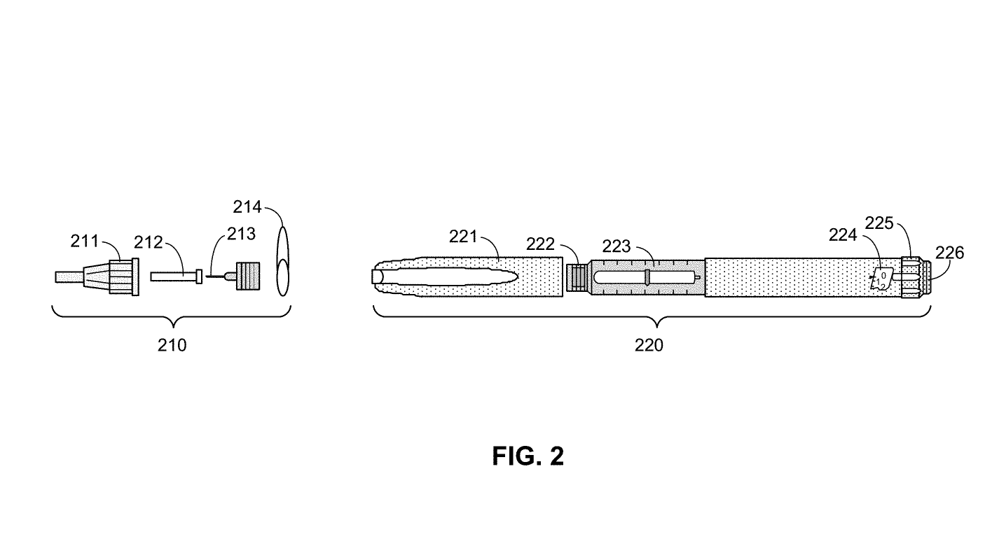

FIG. 2 is an exploded view of a needle assembly 210 and pen injector 220, according to an embodiment. Here, the pen injector 220 (and accompanying needle assembly 210) is a specific type of hypodermic needle device 110, although other types of hypodermic needle devices may have similar components. It will be understood, however, that various other types of hypodermic needle devices (e.g., auto injectors, syringes, etc.) may be utilized in accordance with the techniques described herein, and that the pen injector 220 in FIG. 2, is provided simply an example. Similarly, needle assemblies may also vary from the needle assembly 210 also illustrated in FIG. 2. Moreover, although the needle assembly 210 is illustrated as being separate from the pen injector 220, alternative embodiments of pen injectors or (more broadly) hypodermic needle devices 110 may comprise some or all of a needle assembly incorporated therein. It will also be understood that, in alternative embodiments, the various components of a needle assembly and the hypodermic needle device a may vary in size, shape, and/or other ways from the components of the needle assembly 210 and pen injector 220 illustrated in FIG. 2.

Here, the needle assembly 210 comprises an outer needle cap 211, an inner needle cap 212, a needle 213 (including a base, attachable to the pen injector 220), and a protective seal 214. Because the needle assembly 210 includes the needle 213 that is inserted into the skin of the patient, the needle assembly 210 is typically disposed of after use for sanitary purposes. Thus, a new needle assembly 210 may be used for each injection.

The pen injector 220 stores the drug to be dispensed, and may be reused until the drug is depleted. Here, the pen injector 220 comprises a pen cap 221 that covers an attachment portion 222 to which the needle 213 may be coupled (e.g. by screwing the base of the needle to the attachment portion 222, using force to snap the needle 213 into place, and/or other attachment means). When the needle 213 is coupled to the pen injector 220, the pen cap 221 may be sufficiently large to protect the needle 213.

The pen injector 220 further comprises a reservoir 223 that holds a liquid drug. As described in further detail below, during administration, a piston (not shown) moves through the reservoir 223 to displace a volume of the drug, causing the drug to be dispensed through the needle 213 (when the needle 213 is properly coupled to the pen injector 220).

The pen injector 220 also includes a dosage window to 224 and dose selector 225, enabling a user to select a dose of the drug to be dispensed. The selection can be made by twisting the dose selector 225 (e.g., clockwise or counterclockwise) and selecting a desired dosage, which is shown through the dosage window 224.

Finally, the pen injector 220 includes an injection button 226. Once the needle 213 has been attached to the pen injector 220 and inserted into the skin of a patient, the drug may be administered by the patient (or other user) by pressing the injection button 226, causing a piston to move through the reservoir 223 as indicated above, and pushing a proper dosage of the drug through the needle for administration to the patient.

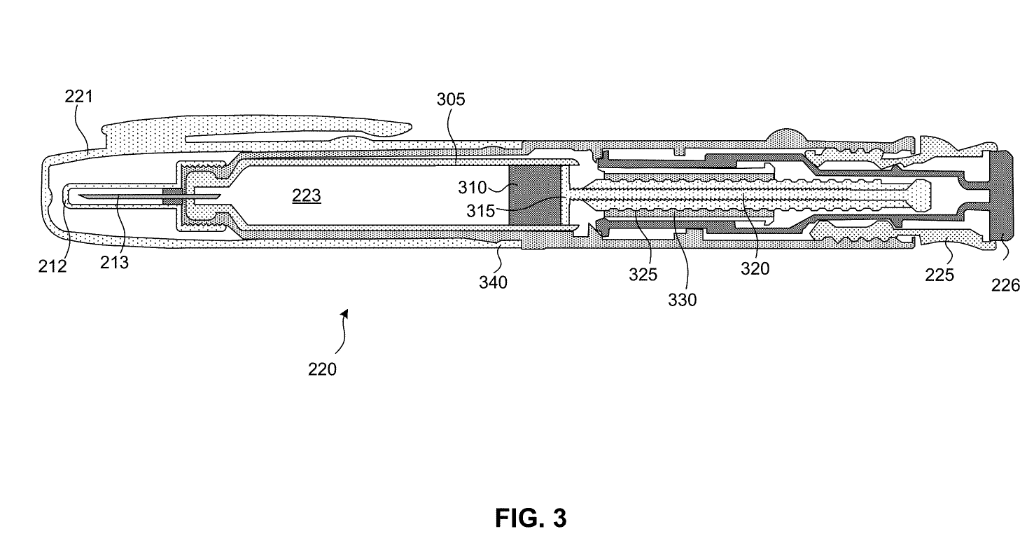

FIG. 3 is a cross-sectional view of a pen injector 220, according to an embodiment. Again, the pen injector 220 is a specific type of hypodermic needle device 110, although other types of hypodermic needle devices may have similar components. However, will also be understood that, in alternative embodiments, the various components of a needle assembly and the hypodermic needle device a may vary in size, shape, and/or other ways from the components of the pen injector 220 illustrated in FIG. 3.

In the illustration in FIG. 3, some components illustrated in FIG. 2 are shown, including the pen cap 221, inner needle cap 212, needle 213, reservoir 223, and injection button 226. The pen injector further includes a cartridge 305 that stores the drug and comprises the reservoir 223. (In some embodiments, the cartridge may be replaceable, enabling the pen injector 220 to be used with multiple cartridges.) During the administration of the drug, a piston 310 is pushed by the head 315 of a drive stem 320, displacing the drug in the reservoir 223 to dispense the drug. The drive stem 320 may be screw driven, having threads 325 that feed the drive stem 320 through a nut 330. When the user presses the injection button 226, the movement of the drive stem 320 and piston 310, and corresponding volume of the drug in the reservoir 223 can be regulated by the dose selector 225.

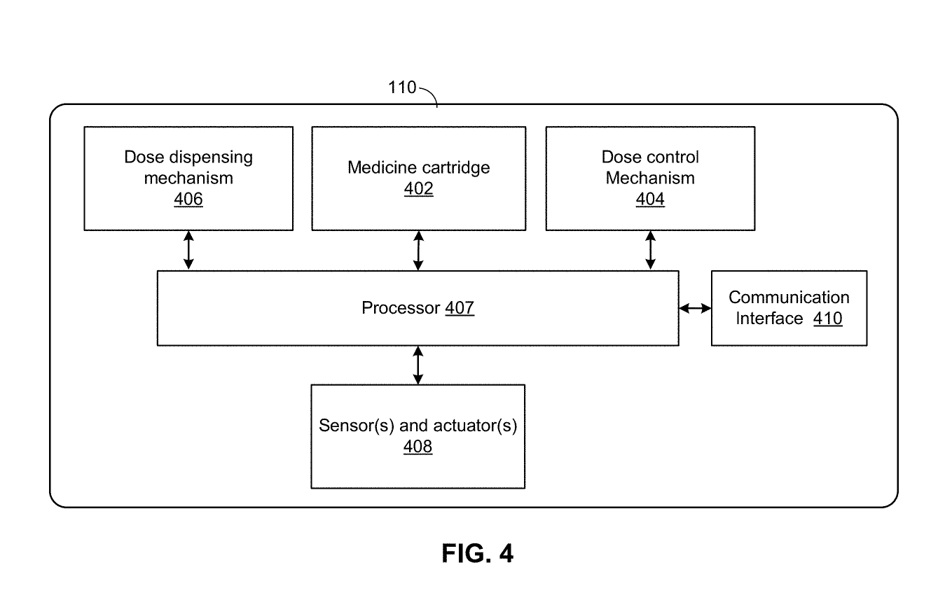

Again, the pen injector 220 illustrated in FIGS. 2-3 is provided as an example hypodermic needle device 110. Techniques for tracking and/or controlling the flow of the drug through the needle during administration of the drug may be applied to other types of hypodermic needle devices 110, including auto injectors, syringes, and the like. A hypodermic needle device 110 may be described more generally as having various components is illustrated in FIG. 4.

FIG. 4 is a block diagram illustrating the of components of a hypodermic needle device 110, according to an embodiment. The hypodermic needle device 110 can include a housing (not shown) structured to hold a medicine cartridge 402 (such as the cartridge 305 of the pen injector 220 illustrated in FIG. 3), which may store the drug to be dispensed by the hypodermic needle device 110. The hypodermic needle device 110 can also include a dose control mechanism 404 to select or set a dose of the drug to be dispensed. The hypodermic needle device 110 further includes a dose dispensing mechanism 406 to dispense a dose of the drug, from medicine cartridge 402, based at least in part on the dose selected or set by dose control mechanism 404.

The hypodermic needle device 110 may include other devices to facilitate dispensing of medicine. In the example of FIG. 4, the hypodermic needle device 110 includes sensor(s) and actuator(s) 408. Additionally, the hypodermic needle device 110 can include a processor 407 communicatively coupled with the sensor(s) and actuator(s) 408 and configured to, among other things, control the operations of the actuator(s) based at least in part on the information collected by the sensor(s). For example, the sensors of sensor(s) and actuator(s) 408 can collect information of certain physical conditions at, for example, medicine cartridge 402, dose control mechanism 404, and dose dispensing mechanism 406. Based at least in part on the collected information, the processor 407 can control the actuators of sensor(s) and actuator(s) 408 to change the operations of dose control mechanism 404 and/or dose dispensing mechanism 406. For example, the sensor(s) and actuator(s) 408 may comprise a flowmeter, and, based at least in part on one or more measurements from the flowmeter, the actuator(s) can be controlled to change the operations of dose control mechanism 404 and/or dose dispensing mechanism 406 to prohibit administration (or further administration) of the drug.

The processor 407 may comprise without limitation one or more general-purpose processors, one or more special-purpose processors (such as digital signal processing (DSP) chips, graphics acceleration processors, application specific integrated circuits (ASICs), and/or the like), and/or other processing structure or means, which can be configured to perform one or more of the methods described herein. To help increase shelf life of the hypodermic needle device 110, the processor 407 may be configured to operate in an extremely low power mode that, along with the capacity of a power supply (not shown), can allow the electrical components of the hypodermic needle device 110 to be used in after a substantially long time of no use. In some embodiments, the processor 407 may utilize additional hardware and/or software components (e.g., a memory) to provide the functionality described herein.

The hypodermic needle device 110 may include a communication interface 410 which can communicate using wireless and/or wired means (e.g., via communication link 120 and/or 125 of FIG. 1). Communication interface 410 may enable transmission of information related to administering the drug, including one or more measurements from a flowmeter and/or an indication of whether the drug was administered properly. Additionally or alternatively, the hypodermic needle device 110 may communicate information related to a quantity of medicine to be dispensed, a quantity of medicine that has been dispensed, a quantity of medicine remaining in medicine cartridge 402, etc. The information can then be displayed to the user via an user interface, to assist the user in administering of the medicine.

As indicated above, techniques described herein may use a flowmeter to measure an actual volume of the amount of drug dispensed to the patient. From architectural standpoint, the flowmeter may compose part of the dose dispensing mechanism 406, sensor(s) and actuator(s) 408, and/or other components of a hypodermic needle device 110 is illustrated in FIG. 4. Alternatively, the flowmeter may comprise part of a separate module. The flowmeter may communicate with hardware and/or software components, including processor 407, which may retrieve data from the flowmeter.

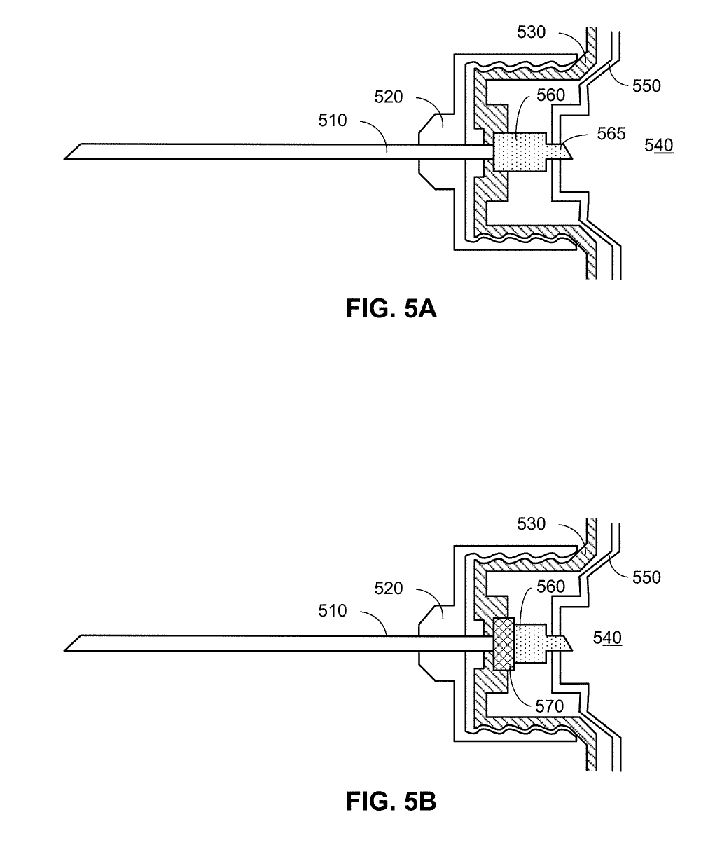

Mechanically, the flowmeter may be situated between the drug cartridge and the needle assembly, held in place within the housing of the hypodermic needle device. Examples of this are illustrated in FIGS. 5A and 5B (which are simplified drawings provided for illustrative purposes).

FIG. 5A is a simplified cross-sectional diagram of a portion of a hypodermic needle device having a flowmeter, according to an embodiment. The cross-sectional diagram illustrates a needle assembly (similar to needle assembly 210 of FIG. 2) having a needle 510 (a hollow tube typically fashioned from stainless steel) secured to a base 520, which is removably attached to the body 530 of the hypodermic needle device (e.g., at attachment portion 222 of a pen injector 220, as illustrated in FIG. 2). (For simplicity, only the portion of the body 530 to which the base 520 is attached as illustrated.) Although illustrated in FIGS. 5A and 5B as using screwing means, the means by which the base 520 is attached to the body 530 can include additional or alternative means (e.g., a snap, latch, etc.), depending on desired functionality. In FIG. 5A, the base forms a female attachment with threads that screw to a corresponding threads of a male attachment of body 530 of the hypodermic needle device. as illustrated in the embodiment of FIG. 3, a cartridge 540, comprising a reservoir of the drug surrounded by a cartridge body 550 may be housed within the body 530 of the hypodermic needle device. As previously noted, in some embodiments, the cartridge 540 may be removable, enabling the hypodermic needle device to be used with multiple cartridges.

According to the embodiments provided herein, a flowmeter 560 may be located between the cartridge 540 and the needle 510, thereby enabling the flowmeter 560 to measure the flow between the cartridge 540 and the needle 510. As such, it can provide a highly accurate determination of how much of the drug from the cartridge 540 is dispensed to the patient. The flowmeter 560 may be held in place (e.g., bonded or otherwise fastened) to the body of the hypodermic needle device communicatively coupled with a processor (e.g., processor 407 of FIG. 4) to enable the processor to receive data from the flowmeter 560 including measurements of the volume of liquid (the drug) that passes through the flowmeter 560. (The flowmeter 560 may be communicatively coupled with the processor via wires and/or wireless means (not shown).) Additionally, depending on desired functionality, the body of the flowmeter 560 may be shaped so that a portion 565 penetrates a seal of the cartridge 540, enabling the drugs to pass from the reservoir of the cartridge 540 through the flowmeter 560 to an opening on the opposite end of the flowmeter into which the needle 510 is inserted. The opening of the flowmeter 560 into which the needle 510 is inserted and/or the portion 565 of the body of the flowmeter 560 that penetrates the cartridge 540 may comprise one or more materials to help ensure a seal is made between the flowmeter, the cartridge 540, and the needle 510, such that the drug does not leak as it passes from the cartridge 540 through the needle 510. That said, alternative embodiments may use flowmeters having different shapes, materials, or other means, other than those illustrated in FIG. 5A for ensuring proper liquid flow from the cartridge 540 to the needle 510

The flowmeter 560 can comprise any of a variety of types of flowmeters, depending on desired functionality. That is, the flowmeter 560 can operate on any of a number of flow measurement principles. In one embodiment, for example, flow measurement by the flowmeter 560 may measure a pressure differential across an orifice or Venturi tube, or cooling effect versus flow rate over a temperature sensor.

According to some embodiments, the flowmeter 560 may comprise a Coriolis flow sensor, which measures both fluid mass flow rate (not volume) and fluid density directly without being influenced by temperature. As such, such embodiments may not need to perform any temperature compensation for measurements taken where fluctuations in temperature may affect the volume of the drug. The Coriolis flow sensor may be appropriately sized for placement between the needle 510 and the cartridge 540. As such, a Coriolis flow sensor may be MEMS-based.

According to some embodiments, where the flowmeter 560 comprises a Coriolis flow sensor, one or more density measurements could be taken to verify the proper drug is used. That is, because the Coriolis flow sensor measures density, it can take one or more density measurements before or during the administration of the drug, to help verify the right drug is being dispensed. For example, a flowmeter 560 may provide a density measurement to a processor during the course of administration of the drug. The processor can then, which compare the density to an average and/or range of densities of an expected drug (e.g., by searching a lookup table stored locally in a memory of the hypodermic needle device, or remotely and communicated to the hypodermic needle device via a communication interface). If the comparison indicates that the wrong drug is being dispensed (e.g., a cartridge 540 of a drug, other than the expected drug, has been inserted into the hypodermic needle device), the processor can cause the hypodermic needle device to provide an indication/warning to the patient that the wrong drug is being dispensed, communicate (e.g., via wireless communication sent from a communication interface (e.g., communication interface 410 in FIG. 4) of the hypodermic needle device, and/or cause the hypodermic needle device to stop dispensing the drug (e.g., by controlling a valve, as indicated in further detail below). (It can be noted that a Coriolis flow sensor may be utilized in medical devices administering fluids, both gases and liquids. And thus, the techniques provided herein may be utilized in the administration of drugs via medical devices other than a hypodermic needle device, such as an inhaler that administers a spray or gas.) To help ensure an identifiable density, drug manufacturers may engineer the drug to be dispensed with a carrier that has a unique density signature easily identifiable by a Coriolis flow sensor.

FIG. 5B is a simplified cross-sectional diagram of a portion of a hypodermic needle device, similar to FIG. 5A. Here, however, in addition to a flowmeter 560, the hypodermic injection device further comprises a flow valve 570 configured to control the flow of the drug from the cartridge 540 to the needle 510. (Although the embodiment illustrated in FIG. 5B illustrates the flow valve 570 located downstream of the flowmeter 560, alternative embodiments may reposition the flow valve 570 and the flowmeter 560 so that the flow valve 570 is upstream of the flowmeter 560.) The flow valve 570 may be attached (e.g., bonded or otherwise fastened) to the body 530 of the hypodermic needle device.

As with the flowmeter 560, the underlying technology of the flow valve 570 may vary, depending on desired functionality. There are a number of operating principles that can be miniaturized for such use in a hypodermic needle device. For example, the flow valve 570 may be operated by a piezoelectric element and/or by an electromagnetic element. According to some embodiments, the flow valve 570 may be in a normally closed position. By applying a voltage to the valve, it can then be opened to allow the drug to flow from the cartridge 540 through the flowmeter 560, and to the needle 510. A processor or other electrical circuitry of the hypodermic needle device may be electronically coupled with the flow valve 570 to provide such voltage. It can be noted that by having a normally closed valve as part of the hypodermic needle device, the hypodermic needle device may have no need for priming.

In some embodiments, the flow valve 570 may have more than a binary output (e.g., either open or closed), providing flow control as well. As such, the flow valve 570 can provide flow control for certain drugs that may need to be dispensed at a certain rate, or where the drugs may need to have a flow rate managed based on other inputs (e.g., environmental, temperature, etc.). In such embodiments, flow control may be provided by controlling a voltage provided to the flow valve 570. And again, a processor (e.g., microcontroller, microprocessor, or similar circuitry) may be used to provide such voltage.

Because a processor can the communicatively coupled with the flowmeter 560 and the flow valve 570 it can use information from the flowmeter 560 to accurately track and control the amount of the drug dispensed to a patient. For example, a processor can receive, as an input, a dose setting. In some embodiments, the dose setting may be input manually by a dose selector, such as the dose selector 225 of the pen injector 220 illustrated in FIGS. 2 and 3. In some embodiments, a dose may be communicated to the hypodermic needle device from another device. For example, in a system such as the one illustrated in FIG. 1, a dose may be selected by a connecting device 130 (e.g., by the patient or another user entering a dosage amount into a graphical user interface shown on a display of the connecting device 130) and/or a device operated by a stakeholder 160 and communicated to the hypodermic needle device 110 via communication links 120 and/or 125. A flow rate (if desired) may be entered in a similar manner. The processor can then use the dose input (and optionally the flow rate) and, based on the patient (or other user) pressing a button or otherwise initiating administration of the drug (e.g., by pressing an end cap attached to the plunger as an indication to start dispensing the drug) the processor could open the flow valve 570 and monitor the amount of drug delivered using the flowmeter 560 until the set drug dose was measured. Once the proper dose was dispensed, the processor could then close the flow valve 570. This functionality could be implemented their respective of the mechanics of the hypodermic injection device used to deliver the drug (e.g., the mechanical aspects of a dose dispensing mechanism 406 of the hypodermic injection device illustrated in FIG. 4) and the variability of those mechanics with temperature or other effects. As the flow valve 570 and flowmeter 560 can therefore compose part of the control loop utilized by the hypodermic needle device to ensure the proper drug is dispensed in the proper way. If it is determined that the wrong drug is used (e.g., a measured density of the drug being dispensed is not within an acceptable range of a drug expected to be dispensed by the hypodermic needle device) the processor controller can immediately suspend the injector operation (e.g., by closing the flow valve 570 and/or suspending other mechanical operations within the hypodermic needle device that dispensed the drug). In such cases, an indicator, alarm, or message can be provided to the patient. In some embodiments, this may be provided by the hypodermic needle device itself (e.g., via an audio alarm provided by a speaker, a visual alarm provided by a display or LED, etc.), and/or communicated to a separate device (e.g., a connecting device 130) that provides the indicator, alarm, or message.

The utilization of a flowmeter 560 and (optionally) a flow valve 570 in the manner described in the embodiments provided above can provide additional or alternative functionality. For example, as noted above, an accurate measurement of an dispensed dosage by the flowmeter 560 may be used to determine whether the drug was dispensed properly. In such embodiments, the dosage measurement taken by the flowmeter 560 may be combined with one or more other types of data (e.g., a timer tracking a length of time during which the drug was dispensed, one or more sensors (e.g., touch, impedance, etc.) configured to determine whether an injection button was pressed by the skin of a user and/or the needle was injected into a patient's skin, etc.) to make the determination of effectiveness of drug administration. This determination and/or the underlying dosage measurement(s) may be sent to a another device, such as a connecting device (e.g., connecting device 130 of FIG. 1) and/or a device of one or more stakeholders (e.g., stakeholder(s) 160).

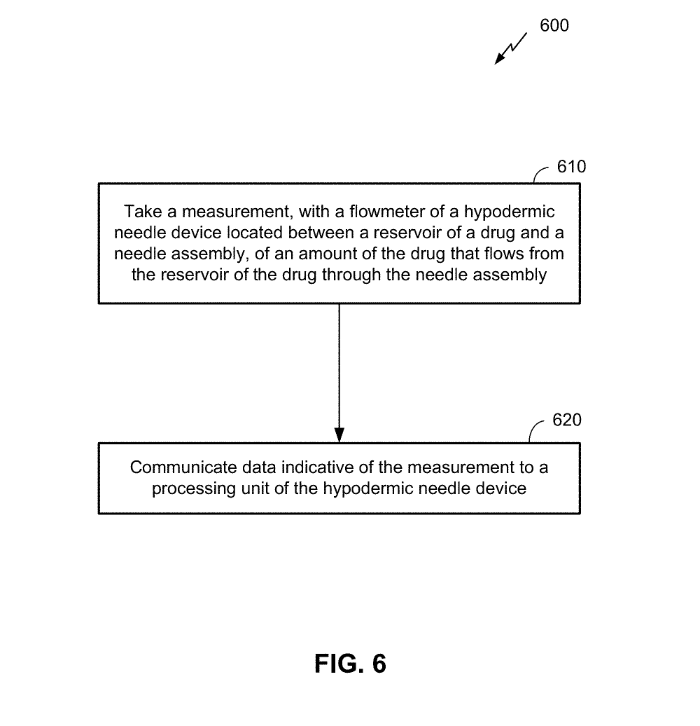

FIG. 6 is a flow diagram illustrating a method 600 of dispensing a drug with a hypodermic needle device, according to an embodiment. It can be noted that, as with figures appended hereto, FIG. 6 is provided as a non-limiting example. Other embodiments may vary, depending on desired functionality. For example, the functional blocks illustrated in method 600 may be combined, separated, or rearranged to accommodate different embodiments. The method 600 may be performed by a hypodermic needle device. Means for performing the functionality of method 600 may include one or more components of the hypodermic needle device, including hardware and/or software components, as illustrated in FIG. 4 above. Hardware components may include analog and/or digital circuitry, including one or more processors, which may be communicatively coupled with a flowmeter and/or flow valve. A person of ordinary skill in the art will appreciate the various means by which the functions in method 600 may be performed.

The functionality at block 610 comprises taking a measurement, with a flowmeter of a hypodermic needle device located between a reservoir of a drug and a needle assembly, of an amount of the drug that flows from the reservoir of the drug through the needle assembly. As noted previously, in some embodiments, the body of the hypodermic needle device may be configured to house a removable cartridge that stores the reservoir of the drug. In such instances, a body of the flowmeter may be shaped to pierce a seal of the removable cartridge when the removable cartridge is inserted into the body of the hypodermic needle device.

Means for performing the functionality of block 610 may comprise, for example, a flowmeter, which may compose part of the sensor(s) and actuator(s) 408, a dose control mechanism 404, and/or other components of the hypodermic needle device 110 illustrated in FIG. 4 and described above.

At block 620, data indicative of the measurement is communicated to a processor of the hypodermic needle device. As previously indicated, a processor may comprise a microcontroller, microprocessor, or similar circuitry configured to receive data from the flowmeter. The processor can further utilize this data in any number of ways, as indicated in the embodiments described above. In some embodiments, for example, the data may be communicated to a separate device using, for example, a communication interface (e.g., communication interface 410 illustrated in FIG. 4) of the hypodermic needle device. In some embodiments, the processor can further control a flow control valve to control the flow of the drug and/or stop administration of the drug, based at least in part on the data indicative of the measurement. Some embodiments may further include sending, to another device, an indication of the determination of the effectiveness of how the drug was dispensed with the hypodermic needle device.

The method 600 may include additional functionality in some embodiments. For example, in some embodiments, the flowmeter may comprise a Coriolis flow sensor. In such embodiments, the Coriolis flow sensor may configured to measure a density of the drug and communicate the measurement of the density of the drug to the processor. The processor may then be configured to, based at least in part on the measurement of the density of the drug received from the Coriolis flow sensor, determine a type of the drug dispensed. If the processor determines the wrong drug is being dispensed, the processor can then stop administration of the drug.

In some embodiments, the hypodermic needle device may further comprise a flow valve coupled with the processor and located within the hypodermic needle device such that, when the drug is being dispensed by the hypodermic needle device, the flow valve is located between the reservoir of the drug and the needle assembly. The flow valve may be a voltage-controlled flow valve. In such cases, the processor may be configured to operate the flow valve to stop the administration of the drug based at least in part on a triggering event. Triggering events can include, for example, a determination (e.g., by the processor) that a predetermined amount of a dosage has been dispensed, a determination (e.g., by the processor) that the wrong drug is being dispensed, based at least in part on a density measurement taken by the flowmeter, or any combination thereof. Additionally or alternatively, the processor may be configured to operate the flow valve using a voltage to control a flow rate of the drug when a drug is being dispensed.

Means for performing the functionality of block 620 may comprise, for example, a processor, which may compose part of the sensor(s) and actuator(s) 408, a dose control mechanism 404, dose dispensing mechanism 406, and/or other components of the hypodermic needle device 110 illustrated in FIG. 4 and described above.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

With reference to the appended figures, components that may comprise memory may comprise non-transitory machine-readable media. The term “machine-readable medium” and “computer-readable medium” as used herein, refer to any storage medium that participates in providing data that causes a machine to operate in a specific fashion. In embodiments provided hereinabove, various machine-readable media might be involved in providing instructions/code to processors and/or other device(s) for execution. Additionally or alternatively, the machine-readable media might be used to store and/or carry such instructions/code. In many implementations, a computer-readable medium is a physical and/or tangible storage medium. Such a medium may take many forms, including but not limited to, non-volatile media, volatile media, and transmission media. Common forms of computer-readable media include, for example, magnetic and/or optical media, any other physical medium with patterns of holes, a RAM, a PROM, EPROM, a FLASH-EPROM, any other memory chip or cartridge, a carrier wave as described hereinafter, or any other medium from which a computer can read instructions and/or code.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein can be embodied in hardware and/or software. Also, technology evolves and, thus, many of the elements are examples that do not limit the scope of the disclosure to those specific examples.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

Therefore, it is intended that claimed subject matter not be limited to the particular examples disclosed, but that such claimed subject matter may also include all aspects falling within the scope of appended claims, and equivalents thereof.

What is claimed is:

1. A hypodermic needle device comprising:
a body;
a flowmeter secured by the body of the hypodermic needle device such that, when a drug is being dispensed by the hypodermic needle device, the flowmeter is located between a reservoir of the drug and a needle assembly, wherein the flowmeter is further configured to take a measurement of an amount of the drug that flows from the reservoir of the drug through the needle assembly; and
a processor communicatively coupled with the flowmeter and configured to receive, from the flowmeter, the measurement of the amount of the drug that flows from the reservoir of the drug through the needle assembly.

2. The hypodermic needle device of claim 1, wherein the flowmeter comprises a Coriolis flow sensor.

3. The hypodermic needle device of claim 2, wherein:
the Coriolis flow sensor is configured to take a measurement of a density of the drug and communicate the measurement of the density of the drug to the processor; and
the processor is configured to, based at least in part on the measurement of the density of the drug received from the Coriolis flow sensor, determine a type of the drug dispensed.

4. The hypodermic needle device of claim 1, further comprising a flow valve communicatively coupled with the processor and located within the hypodermic needle device such that, when the drug is being dispensed by the hypodermic needle device, the flow valve is located between the reservoir of the drug and the needle assembly.

5. The hypodermic needle device of claim 4, wherein the processor is configured to operate the flow valve to stop administration of the drug based at least in part on a triggering event.

6. The hypodermic needle device of claim 5, wherein the triggering event comprises:
a determination that a predetermined amount of a dosage has been dispensed, or
a determination that a wrong drug is being dispensed, based at least in part on a density measurement taken by the flowmeter, or
any combination thereof.

7. The hypodermic needle device of claim 4, wherein the processor is configured to operate the flow valve using a voltage to control a flow rate of the drug when a drug is being dispensed.

8. The hypodermic needle device of claim 1, wherein the body of the hypodermic needle device is further configured to house a removable cartridge, wherein the removable cartridge stores the reservoir of the drug.

9. The hypodermic needle device of claim 8, wherein a body of the flowmeter is shaped to pierce a seal of the removable cartridge when the removable cartridge is inserted into the body of the hypodermic needle device.

10. A method of dispensing a drug with a hypodermic needle device comprising:
taking a measurement, with a flowmeter of a hypodermic needle device located between a reservoir of the drug and a needle assembly, of an amount of the drug that flows from the reservoir of the drug through the needle assembly; and
communicating data indicative of the measurement to a processor of the hypodermic needle device.

11. The method of claim 10, wherein the flowmeter comprises a Coriolis flow sensor.

12. The method of claim 11, further comprising:
measuring, with the Coriolis flow sensor, a density of the drug;
communicating the measurement of the density of the drug to the processor of the hypodermic needle device; and
determining, with the processor of the hypodermic needle device, a type of the drug dispensed, based at least in part on the measurement of the density of the drug.

13. The method of claim 10, wherein the hypodermic needle device further comprises a flow valve communicatively coupled with the processor and located within the hypodermic needle device such that, when the drug is being dispensed by the hypodermic needle device, the flow valve is located between the reservoir of the drug and the needle assembly.

14. The method of claim 13, further comprising operating the flow valve to stop administration of the drug based at least in part on a triggering event.

15. The method of claim 14, wherein the triggering event comprises:
a determination that a predetermined amount of a dosage has been dispensed, or
a determination that a wrong drug is being dispensed, based at least in part on a density measurement taken by the flowmeter, or
any combination thereof.

16. The method of claim 14, wherein operating the flow valve comprises using a voltage to control a flow rate of the drug when a drug is being dispensed.

17. The method of claim 10, wherein:
a body of the hypodermic needle device is further configured to house a removable cartridge; and
the removable cartridge stores the reservoir of the drug.

18. The method of claim 17, wherein a body of the flowmeter is shaped to pierce a seal of the removable cartridge when the removable cartridge is inserted into the body of the hypodermic needle device.

19. A hypodermic needle device comprising:
measuring means configured to take a measurement of an amount of a drug that flows from a reservoir of the drug within the hypodermic needle device through a needle assembly of the hypodermic needle device, wherein the measuring means is located between the reservoir the needle assembly; and
processing means configured to obtain data indicative of the measurement from the measuring means.

20. The hypodermic needle device of claim 19, wherein the measuring means comprises a Coriolis flow sensor.

21. The hypodermic needle device of claim 20, wherein:
the measuring means is configured to measure a density of the drug; and
the processing means is configured to:
obtain the measurement of the density of the drug; and
determine a type of the drug dispensed, based at least in part on the measurement of the density of the drug.

22. The hypodermic needle device of claim 19, further comprising flow restriction means communicatively coupled with the processing means and located within the hypodermic needle device such that, when the drug is being dispensed by the hypodermic needle device, the flow restriction means is located between the reservoir of the drug and the needle assembly.

23. The hypodermic needle device of claim 22, further comprising means for operating the flow restriction means to stop administration of the drug based at least in part on a triggering event.

24. The hypodermic needle device of claim 23, wherein the triggering event comprises:
a determination that a predetermined amount of a dosage has been dispensed, or
a determination that a wrong drug is being dispensed, based at least in part on a density measurement taken by the measuring means, or
any combination thereof.

25. The hypodermic needle device of claim 23, wherein the means for operating the flow restriction means comprises means for using a voltage to control a flow rate of the drug when a drug is being dispensed.

26. The hypodermic needle device of claim 19, further comprising means for housing a removable cartridge that stores the reservoir of the drug.

27. The hypodermic needle device of claim 26, further comprising means for piercing a seal of the removable cartridge when the removable cartridge is inserted into the hypodermic needle device.

28. A non-transitory computer-readable medium comprising instructions embedded thereon for dispensing a drug with a hypodermic needle device, the instructions comprising computer code for:
taking a measurement, with a flowmeter of a hypodermic needle device located between a reservoir of the drug and a needle assembly, of an amount of the drug that flows from the reservoir of the drug through the needle assembly; and
communicating data indicative of the measurement to a processor of the hypodermic needle device.

29. The non-transitory computer-readable medium of claim 28, wherein the instructions further comprise computer code for operating a flow valve to stop administration of the drug based at least in part on a triggering event.

30. The -transitory computer-readable medium of claim 29, wherein the instructions further comprise computer code for determining the triggering event, the instructions for determining the triggering event comprising:
instructions for determining that a predetermined amount of a dosage has been dispensed,
instructions for determining that a wrong drug is being dispensed, based at least in part on a density measurement taken by the flowmeter, or
any combination thereof.
